(19) United States
(12) Patent Application Publication
(10) Pub. No.: US 2006/0057021 A1
(43) Pub. Date: Mar. 16, 2006

(54) Fixed vacuum-insulated saturated steam autoclave

(76) Inventors: Melvyn Lloyd Sawyer, Vancouver (CA); Nigel Halsted, Burnaby (CA)

Correspondence Address: KIRBY EADES GALE BAKER, BOX 3432, STATION D, OTTAWA, ON, K1P 6N9, CA

(21) Appl. No.: 11/221,735

(22) Filed: Sep. 9, 2005

Related U.S. Application Data
(63) Continuation-in-part of application No. 10/270,428, filed on Oct. 15, 2002.

(51) Int. Cl. A61L 2/07

(52) U.S. Cl. 422/26; 422/292

(57) ABSTRACT

A portable table-top steam sterilizer having a sterilization chamber contained within a double-walled vacuum-sealed vessel. Pre-heated water is delivered from a self-contained water supply infrastructure under a positive air pressure to the chamber. The pre-heated water is converted to steam within the sterilization chamber for a sterilization cycle after which, the steam is rapidly removed by vacuum and the sterilization chamber and items placed therein are rapidly cooled and dried by a turbulent flow of air. The air supply is provided by concurrent application of positive and negative air pressures from a self-contained airflow piping infrastructure releasably engaged with an external supply of compressed air. The mixtures of steam, water and air exhausted from the sterilization chamber are directed into a condenser wherein they are cooled into an air/water mixture which is transferred to an air/water separator device wherein water is separated from the air/water mixture, said water recyclable within the self-contained water supply infrastructure.

### CROSS-REFRFENCE TO RELATED APPLICATION

This application is a continuation-in-part of our prior application Ser. No. 10/270,428, filed Oct. 15, 2002, currently pending.

### FIELD OF THE INVENTION

This invention relates to steam sterilizers. More particularly, this invention relates to portable table-top steam sterilizers suitable for sterilizing therein dental or medical or veterinary instruments and supplies.

### BACKGROUND OF THE INVENTION

Table-top steam sterilizers, commonly referred to as autoclaves, are widely used for the sterilization of instruments and other articles used in the medical, dental and veterinary disciplines. Contents of such autoclaves, when exposed to the proper conditions of temperature, time and pressure within saturated steam environments provided therein during sterilization runs, will be rendered sterile, i.e., free of any microorganisms and viruses that may have been present on the articles prior to the sterilization operation.

In contrast with large fixed-in-place hospital-type steam sterilizers that are fed steam from a separate steam source, table-top autoclaves generally are portable, heated pressure vessels that generate their own steam. They are small enough to fit onto countertops commonly present in dental and medical laboratories and offices. These types of autoclaves typically have a small cylindrical sterilization chamber provided with a single door and latch assembly.

The operation of such table-top autoclaves generally proceeds as follows. First, pre-cleaned articles to be sterilized are placed into the autoclave and the door is closed and latched shut. Then, the chamber is heated by the direct application of heating elements to the outer bottom surface of the chamber, and then a small amount of water is released from a self-contained water reservoir into the preheated chamber. As the water in the chamber is heated to the boiling point, steam is formed. The steam, being lighter than air, rises to the top of the chamber, and thus displaces cooler air to the bottom of the chamber. This cooler, unsaturated air then evacuates through an open valve. Once the temperature of the chamber reaches a preset value, the chamber is deemed to be saturated with steam and the open valve closes. Pressure and temperature then build in the closed chamber until a predetermined temperature and pressure level are reached. These levels are then held for a prescribed period of time to enable complete destruction of microorganisms, spores and viruses inside the chamber and on the contents therein, rendering a sterile condition. Steam is then released from the chamber and the contents of the chamber are removed.

There are certain well-known and previously described problems that occur with autoclaves. The sterilization chamber, or pressure vessel, must be free of air and filled with saturated steam during the sterilization cycle. Failure to do so could result in pockets of air remaining in the sterilizer, resulting in cold spots where articles would not be subjected to sterilizing conditions. As well, water condensation could occur inside the chamber, also potentially entrapping microorganisms resulting in sterilization failure. Suggested solutions to these problems have included the use of a pre-sterilization vacuum applied to the chamber to remove air prior to steam injection, as well as using sophisticated temperature and pressure measurements conforming to conventional pre-set values which assume air removal from the chamber. The use of a vacuum pump to evacuate air from the chamber adds additional expense and increased potential for mechanical breakdown of the autoclave. The use of temperature and pressure sensors that are controlled by microprocessors to respond to predetermined set values also add additional expense to the autoclave. Also, relying on preset and predetermined temperature and pressure values does not take into account variations in atmospheric pressure that exist when the autoclave is operated at altitudes other than sea level.

Another problem that occurs with autoclaves is that it becomes difficult to precisely control the temperature of the chamber, whether the technique of steam injection or steam generation within the chamber via direct application of heating elements are used. Precise temperature control is not only necessary for the assurance of sterility, but it is also critical to ensure that temperatures do not exceed the maximum sterilization temperature that manufacturers recommend for certain temperature sensitive articles. The ability to control temperatures to +/−2 degrees in a pressurized chamber at about 270 degrees Fahrenheit (132 degrees Centigrade) is extremely difficult. This difficulty can be due to heat loss through the chamber wall, the residual heat capacity of the sterilizers beating elements as they are intermittently turned on and off during the sterilization cycle, or due to a rapid rise in the chamber temperature after injection of steam.

In the case of sterilizers that generate their own steam by beating water in the chamber via a direct application of a heating element to the chamber, such heating elements generally have an on/off application of power. During the sterilization cycle, when the sterilizer cools below the set-point, the heating element is turned on at maximum power. Once the temperature reaches the set point, the power to the element is turned off. This difference in temperature is known as the hysteresis. However, due to the on/off application of power, and the residual heat capacity of the heating element, temperature fluctuation inside the sterilizer is generally greater than the hysteresis. When this occurs, chamber temperature overshoots the set point, and sterilization temperatures can exceed manufacturers maximum sterilization temperature tolerances for instruments.

Feathers et al. (U.S. Pat. No. 5,164,161) disclose the use of a proportional controller which applies beat to a number of beaters placed on a large, 58 kilogram sterilizer chamber. This controller measures the temperature differential between various areas on the chamber wall surface and a set point predetermined by a chosen sterilization cycle. The controller applies electricity, and thus heat, proportionally to the surfaces that require an increased amount of heat. This size of sterilizer requires a prolonged warm-up phase, where maximum power is directed to all the heaters. As the described sterilizer also employs an optional unsaturated chemical sterilization cycle, where the temperature inside the chamber is not necessarily constant throughout, it is important to have regional control over the heating process.

Breach discloses in his U.S. Pat. No. 5,858,304 steam injection-type sterilizer provided with a vacuum jacket surrounding the sterilization chamber. A large open space is created between the outer wall of a rectangular shaped sterilizer chamber and an outer shell or jacket. A vacuum is created in this demarcated space, using a vacuum pump which is employed prior to, and then intermittently throughout the sterilization cycle. Steam can also be injected into this space, which helps to keep the steam that is injected into the sterilizer hot for as long a time as possible. The main drawback of this approach is that the sterilization cycle is extended by the amount of time needed to pump down the insulating space, which can add a significant delay. Other drawbacks of such a system are that a separate vacuum pump is required, with adds expense to the autoclave and adds the potential for mechanical breakdown. The noise created by the vacuum pump as well as the space required for the pump and steam generator precludes a table top autoclave design. Another drawback to the system is that it relies on the chamber warm-up coils to keep the chamber in a semi-ready state between sterilization cycles, thus expending electricity when the unit is essentially not in use. Since this type of sterilizer requires steam to be injected into the chamber, the chamber wall must be more than 212° F. (i.e., 100° C.) so that the steam does not condense on the chamber wall when injected into the chamber. Still another drawback to the system is that the door to the sterilizer chamber is not surrounded by a vacuum, consequently providing a large area of heat loss.

### SUMMARY OF THE INVENTION

The present invention, at least in preferred forms, provides a portable table-top steam sterilizer for sterilizing therein dental, medical and veterinary instruments and materials.

According to one aspect of the present invention, there is provided a double-walled vacuum-sealed vessel containing therein a sterilization chamber wherein water is delivered from a self-contained water supply infrastructure, said water heated and converted to steam therein the sterilization chamber for a sterilization cycle after which, the steam is rapidly removed by vacuum and the sterilization chamber and items placed therein are rapidly cooled and dried by a turbulent flow of air provided therethrough by concurrent application of positive and negative air pressures from a self-contained airflow piping infrastructure releasably engaged with an external supply of compressed air.

According to another aspect of the present invention, there is provided a self-contained water supply system interconnected with a heat-conductive metal tubing contiguously wrapped around the outer wall of the double-walled vacuum-sealed vessel, one end of said tubing connected to the water supply infrastructure and the other end of said metal tubing communicating with the sterilization chamber contained therewithin the double-walled vacuum-sealed vessel.

According to yet another aspect of the present invention, there is provided an electric heating element interposed the double-walled vacuum insulated vessel whereby the heating element concurrently heats the sterilization chamber and water contained within the heat-conductive metal tubing.

According to a further aspect of the present invention, there is provided a first airflow piping system for providing a positive air pressure thereto the metal tubing.

According to an additional aspect of the present invention, there is provided a second airflow piping system equipped with a venturi device for providing a negative air pressure thereto the sterilization chamber. The venturi device is adapted for receiving therein in mixtures of steam, water and air exhausted from the sterilization chamber. The venturi device is interconnected with a condenser wherein the steam, water and air mixtures exhausted from the sterilization chamber are cooled.

According to another aspect of the present invention, there is provided an air/water separation apparatus interconnected with the condenser for separating therein water from the cooled air/water mixtures. The separated water is recyclable into the water supply infrastructure.

In a preferred form, the present invention provides a double-walled vacuum-sealed vessel comprising an inner cylindrical container defining a sterilization chamber therewithin, said inner cylindrical container inserted within an outer jacket wherein the mouth of the inner container is hermetically scaled to the mouth of the outer jacket under a negative pressure. The outer surface of the inner cylindrical container and the inner surface of the outer jacket define an annular space therebetween, said annular space having a permanent negative air pressure therewithin thereby providing insulation against heat transfer between the sterilization chamber and the outer jacket.

In another preferred form, the present invention provides an electric heating element fixed to a bottom portion of the outer surface of the inner cylindrical container of the double-walled vacuum-insulated vessel for applying heat directly to the inner wall of said vessel thereby heating the water delivered therein and converting said water to steam within the sterilization chamber. The outer jacket of the double-walled vacuum-sealed vessel is heated by heat radiating from the electric heating element securely fixed to the inner cylindrical container, said radiated heat transferable from the outer jacket to the heat-conductive metal tubing wrapped therearound thereby preheating water contained therein before said water is delivered to the sterilization chamber.

In yet another preferred form, the present invention provides a PID temperature controller communicating and cooperating with a PIC microprocessor for precisely controlling temperatures within said sterilization chamber during a sterilization run, wherein the PID temperature controller communicates with a thermocouple and cooperates with the electric heating element, and the PIC microprocessor communicates with the sterilization chamber and cooperates with the water supply infrastructure and the airflow piping infrastructure.

### BRIEF DESCRIPTION OF THE DRAWINGS

The present invention will be described in conjunction with reference to the following drawings, in which:

FIG. 1 is a cross-sectional side view of one embodiment of the present invention;

FIG. 2 is a cross-sectional end view of the embodiment shown in FIG. 1;

FIG. 3 is a cross-sectional side view of another embodiment of the present invention;

FIG. 4 is a schematic diagram of another embodiment of the present invention; and

FIG. 5 is a schematic diagram of a further embodiment of the present invention.

### DETAILED DESCRIPTION OF THE INVENTION

A preferred embodiment provides a portable table-top steam sterilizer comprising a sterilization chamber having a volume selected from the range of 35.4-70.8 L (i.e., 1.25-2.5 cu. ft.), the sterilization chamber being contained within a double-walled vacuum-sealed vessel having a horizontal longitudinal axis, wherein water is delivered from a self-contained water supply infrastructure, the water being heated and converted to steam in the sterilization chamber for a sterilization cycle, after which, the steam is rapidly removed by vacuum and the sterilization chamber and items placed therein are rapidly cooled and dried by a turbulent flow of air provided therethrough by concurrent application of positive and negative air pressures from a self-contained airflow piping infrastructure releasably engaged with an external supply of compressed air.

Heat-conductive metal tubing for receiving water therein is wrapped around the outer wall of the double-walled vacuum-sealed vessel, one end of the tubing being connected to the water supply infrastructure and the other end of the metal tubing communicating with the sterilization chamber contained within the double-walled vacuum-sealed vessel. An electric heating element is fixed to a bottom portion of the outer surface of the inner wall of the double-walled vacuum-insulated vessel for applying heat directly to the inner wall of the vessel thereby heating the water delivered therein and converting the water to steam for sterilizing items placed on a shelf in the sterilization chamber. If so desired, a heat-conductive metal member may be mounted on the inner surface of the inner wall opposite the electric heating element for increasing the speed of converting water to steam within the sterilization chamber. The outer wall of the double-walled vacuum-sealed vessel is heated by heat radiating from the electric heating element securely fixed to the inner wall, the radiated heat being transferable from the outer wall of the double-walled vacuum-sealed vessel to the heal-conductive metal tubing wrapped therearound, thereby preheating water contained therein before the water is delivered to the sterilization chamber. The self-contained airflow piping infrastructure is configured to provide two high-velocity compressed air flow systems, wherein the first airflow system communicates with the water supply infrastructure to provide air pressure for propulsion of water contained in the metal tubing from the metal tubing into the sterilization chamber, and alternatively, for delivering a high-velocity compressed airflow through said metal tubing into the sterilization chamber for cooling and drying sterilized items contained therein. The second high-velocity airflow system is provided with a venturi device for passing therethrough a high-velocity air flow, thereby providing negative pressures, i.e. a vacuum, to the sterilization chamber through: (a) a vacuum line having one end communicating with the sterilization chamber for removing steam and air therefrom and the other end of the vacuum line having a venturi tube portion interconnected with the venturi device for discharging the steam and air thereinto, and (b) a drain line having one end communicating with the sterilization chamber for removing water therefrom and the other end of the drain line having a venturi tube portion interconnected with said venturi device for discharging said water thereinto. The venturi device is connected to a condenser unit for receiving and cooling therein high-velocity mixtures of compressed air and water and/or compressed air and water and steam discharged from the venturi device. The condenser unit is interconnected with an air/water separation apparatus for slowing the velocity of the air/water mixture entering therein, thereby separating the water from the air, and discharging the water and the air from the air/water separation apparatus. The discharged water is preferably recycled to the water supply infrastructure for reuse, or alternatively, the discharged water may be routed to an external waste water line. The temperature within the sterilization chamber may be precisely controlled and maintained during a sterilization cycle by the communication and cooperation of a PID temperature controller and a PIC microcontroller, the PID temperature communicating with a thermocouple and controllably cooperating with the electric heating element, the PIC microcontroller communicating with humidity and pressure sensors and controllably cooperating with solenoid and check valves integral to the water supply infrastructure and the airflow piping infrastructure, said PIC microcontroller interconnected with electronic user input and output devices.

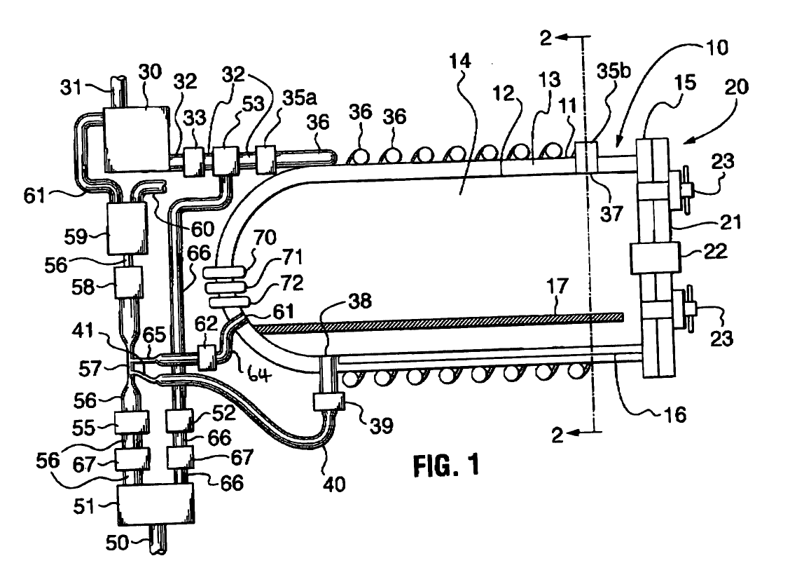

An example of a preferred embodiment of the present invention is shown in FIGS. 1, 2 and 3 wherein double-walled vacuum-sealed vessel 10 comprises an inner cylindrical container 12 defining a sterilization chamber 14 therewithin, said inner cylindrical container being inserted within an outer jacket 11 wherein the mouth of the inner container is hermetically sealed to the mouth of the outer jacket under a negative pressure (vacuum), the seal being provided with a flange 15 protruding beyond the circumference of the outer jacket 11. The inner 34) cylindrical container 12 and outer jacket 11 are preferably manufactured from high tensile strenght stainless steel. The outer surface of the inner container 12 and the inner surface of the outer jacket 11 define an annular space 13 therebetween having a permanent negative air pressure, thereby providing insulation against heat transfer between sterilization chamber 14 and outer jacket 11. It is preferable that the width of annular space 13 between inner cylindrical container 12 and outer jacket 11 is selected from the range of 5 mm to 20 mm. The inner surfaces of inner cylindrical container 12 and outer jacket 11 may optionally be highly polished to mirror-like finishes to facilitate their reflection of heat radiation thereby minimizing radiation heat losses through the stainless steel walls.

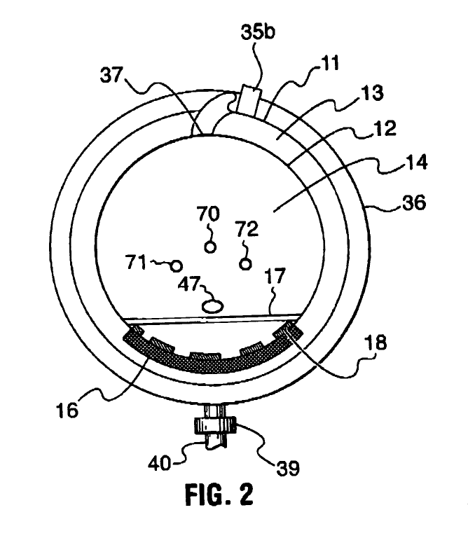

Double-walled vacuum-sealed vessel 10 is provided with a conduit 37 for the flow of water and/or compressed air therethrough, the conduit 37 communicating with a water inlet port (not shown) projecting from the outer jacket 11, a vacuum conduit 61 for application of negative air pressure therethrough, the vacuum conduit 61 communicating with a vacuum inlet port (not shown) projecting from the outer jacket 11, and a drain conduit 38 for draining water therethrough, the drain conduit communicating with a water outlet port (not shown) projecting from the outer jacket 11, the conduits 37, 38, 61 being hermetically scalingly engaged with the inner cylindrical container 12 and the outer jacket 11. An electric heating element 16 in the form of a flexible grid preferably rated at 1,500 watts is securely fixed against the outer surface of the bottom portion of the inner cylindrical container 12 and extends from the front to the rear of the outer surface for controllably heating sterilization chamber 14 contained within inner cylindrical container 12. It is preferable that the electric heating element 16 does not extend beyond the bottom third of the circumference of the cylindrical inner cylindrical container 12. An example of a suitable electric heating element is a silicone-type rubber heater having a wire-wound electrical heating element, or alternatively an etched foil electrical heating element, vulcanized between two layers of silicone rubber or fiberglass, as exemplified by the silicone rubber heaters commercially available from Watlow Electric Manufacturing Company of 2101 Pennsylvania Drive, Columbia, Mo., USA. A plurality of copper bars 18 (see FIG. 2) is mounted on the inner surface of cylindrical container 12 opposite electric heating element 16. The double-walled vacuum-sealed vessel 10 is further provided with a thermocouple 70, a pressure sensor 72, and a humidity sensor 71 for sensing temperature, pressure and humidity changes within the sterilization chamber 14. The sterilization chamber 14 is provided with a horizontal perforated shelf 17 extending from the front to the rear of the chamber 14 for placing thereon items to be sterilized. If so desired, a plurality of horizontal perforated shelves may be provided for placing thereon items to be sterilized. Those skilled in this art will understand that a wide variety of suitable materials may be selected for manufacture of shelf 17 including, for example, perforate sheet metal material, an interwoven metal gridwork engaged with a reinforcing outer metal frame, or an outer metal frame engaged therewith a plurality of spaced apart metal rods for placing thereon items to be sterilized.

Flange 15 extending from the hermetic seal at the mouth of the double-walled vacuum-sealed vessel 10 is provided with hinge 22 for pivotably mounting a stainless steel sterilizer door 20 and one or more latches 23 for sealably and releasibly engaging the door 20 with the mouth of the double-walled vacuum-sealed vessel 10. Stainless steel door 20 may be optionally provided with a double-walled vacuum-sealed chamber which is juxtapositioned to the open end of vessel 10 when door 20 is closed. One or more electromechanical safety devices (not shown) may optionally be provided for communicating and/or cooperating therewith latches 23 to prevent opening of door 20 during the course of a sterilization cycle.

According to a particularly preferred embodiment, the table-top steam sterilizer is provided with a self-contained water supply infrastructure comprising a water reservoir 30, which is optionally connectable to an external water supply line 31, a water outlet line 32 connecting reservoir 30 with one end of a heat-conductive metal tubing 36 wrapped around the outer jacket 11 of double-walled vacuum-insulated vessel 10, the other end of the metal tubing 36 being sealingly engaged with the water inlet port. Water outlet line 32 is provided with water solenoid valve 33 and a first water check valve 35a for controlling the flow of water from the reservoir 30 into the metal tubing 36, and a second water check valve 35b for controlling the flow of water from the metal tubing 36 into the sterilization chamber 14. A pressure-relief valve (not shown) is interposed between the second water check valve 35b and the water inlet port. A water filter (not shown) may optionally be interconnected between the external water supply line 31 and the reservoir 30, or alternatively, between the water reservoir 30 and the water solenoid valve 33. The sterilizer is further provided with an airflow piping infrastructure for providing negative air flows, i.e., a plurality of vacuums, to the sterilization chamber 14 and a positive airflow to the water outlet line 32, the airflow piping infrastructure being connectable to an external compressed air supply 50. The airflow piping infrastructure comprises a main regulator valve 51 connectable to the external compressed air supply 50 for regulating the flow of air into a first high-velocity airflow system and a second high-velocity airflow system. The first high-velocity airflow system is provided with piping 66 connecting the main regulator valve 51 with the water outlet line 32 for providing a high-velocity positive air flow to the water outlet line 32, the air flow being controllable by inline air solenoid valve 52 and an air check valve 53 interposed between water solenoid valve 33 and first water check valve 35a. Air filter 67 for sterilizing the flow of air through piping 66 is interposed between the main regulator valve 51 and air solenoid valve 52. The second high-velocity airflow system comprises piping 56 connected to venturi device 57 feeding into a condenser unit 58 interconnected with an air/water separation apparatus 59 equipped with air exhaust line 60 and water outflow line 61. An air solenoid valve 55 is interposed between main regulator valve 51 and venturi device 57. A vacuum air line 64 is provided wherein one end is sealably engaged with the vacuum inlet port (not shown) provided on outer jacket 11 of double-walled vacuum-sealed vessel 10 for providing a negative air pressure i.e., vacuum therethrough, and the other end of the vacuum air line 64 is provided with a venturi tube portion 65 which is interconnected with venturi device 57 of the second airflow system, the vacuum air line 64 being provided with a vacuum solenoid valve 62 interposed in front of venturi tube portion 65. A drain line 40 is provided with one end engaged with the drain outlet port provided on the outer jacket 11 of double-walled vacuum-sealed vessel 10, and the other end being provided with a venturi tube portion 41 which is interconnected with venturi device 57 of the second airflow system, the drain line 40 being provided with a drain solenoid valve 39 interposed in front of drain line venturi tube portion 41. Drain line 40 may be optionally provided with a diverter valve (not shown) connected to a waste water outlet for by-passing drain line venturi tube portion 41.

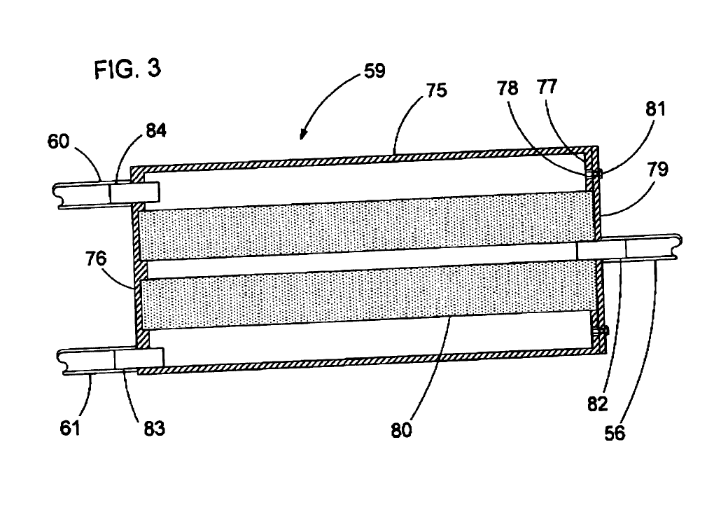

As shown in FIGS. 1 and 3, air/water separator 59 comprises a durable cylindrical container 75 having a closed end 76 configured to receive therein a high-volume flow-through water filter 80, the cylindrical container 75 having an opposite open end provided with rim portion 77 fitted with a plurality of retainer nuts 78. Cap 79 is sealingly secured to cylindrical container 75 by fasteners 81 releasably engaging retainer nuts 78. Cap 79 is provided with inlet port 82 for receiving therethrough the high-velocity air-water mixture exiting from condenser 58 via piping 56. Once inside cylinder 75, the air-water mixture is diffused through high-volume flow-through water filter 80 thereby significantly slowing the velocity of the airflow and separating water from the air-water mixture. The separated water is pulled by gravity to the bottom of cylindrical container 75 from which it is forced by air pressure through a water exit port 83 into water outflow line 61, which is interconnected with water reservoir 30, or alternatively, may empty into an external water waste line (not shown), or optionally, may be interconnected with the water reservoir 30 and also be provided with a diverter valve (not shown) connected with piping (not shown) emptying into an external water waste line (not shown). The excess air is released from air/water separator 59 through an air exit port 84 into air exhaust line 60.

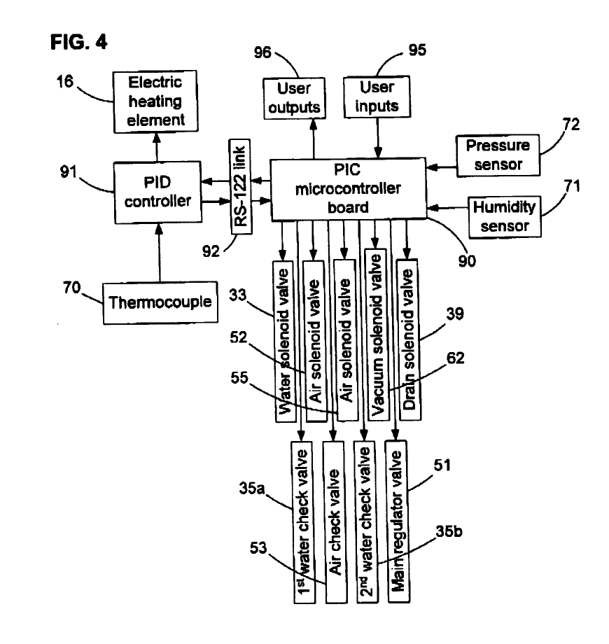

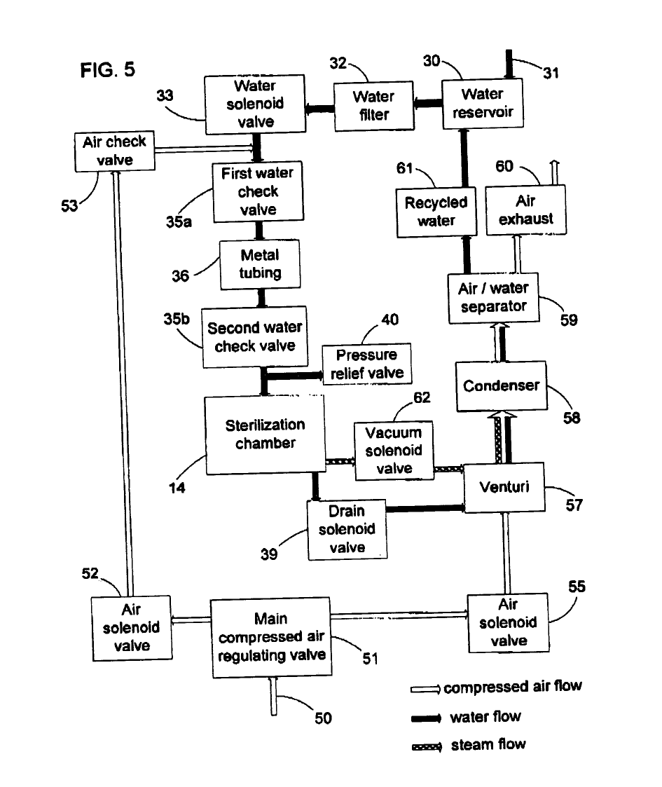

According to another preferred embodiment, the electronic control of the operation of the portable table-top steam is shown in FIGS. 1, 4 and 5 wherein user input electronic device 95 is provided for receiving therein user inputs for setting the duration of sterilization cycles and for starting sterilization runs. The devices 95 communicate with PIC microprocessor 90, which serves as the main electronic controller for communicating with humidity sensor 71 and pressure sensor 72 for monitoring the progress and status of sterilization runs. The progress is displayed on user output electronic device 96. The PIC microprocessor 90 also communicates with solenoid valves 33, 39, 52, 55, 62, check valves 35a, 35b and 53, and main regulator valve 51. Precise heating and control of temperature within sterilization chamber 14 is provided by a PID temperature controller 91 communicating with a thermocouple 70 and electrical beating element 16, in cooperation with PIC microprocessor 90 via a RS-122 link 92. To begin a sterilization cycle, items to be sterilized are placed on shelf 17 and then door 20 is closed and locked by latches 23.

The sterilization cycle is initiated by entering a selected time period for the duration of sterilization and then entering a start command into user input electronic device 95. When the main power switch (not shown) is activated, PIC microcontroller 90 initiates a standby mode wherein drain solenoid valve 39 and vacuum solenoid valve 62 are held in open positions, while water solenoid valve 33, first and second water check valves 35a and 35b, air solenoid valves 52 and 55, and air check valve 53 are held in closed positions, and a stand-by temperature value of, for example, 80° C. is sent electronically by PIC microcontroller 90 to PID temperature controller 91. PID temperature controller 91 then activates electric heating element 16 and monitors the temperature within sterilization chamber 14 by communicating with thermocouple 70. After the standby temperature is reached, an electronic signal is communicated by PID temperature controller 91 to PIC microcontroller 90, whereupon PIC microcontroller 90 activates an electronic signal in user output electronic device 96 indicating that a sterilization run can be commenced. The run is then initiated by an appropriate input into user input device 95. Upon initiation of the sterilization cycle, PIC microcontroller 90 closes drain solenoid valve 39, and opens water solenoid valve 33 and first water check valve 35a until metal tubing 36 is filled with water from reservoir 30, after which, water solenoid valve 33 and first water check valve 35a are closed, and power is supplied to electric heating element 16 by PID temperature controller 91 to produce a temperature set point of preferably 131° C. It is to be noted that the inner diameter and the length of tubing 36 should be selected to contain therein, between the first and second water check valves 35a and 35b, a volume of water required for conversion into steam in chamber 14 during the sterilization cycle. For example, 7.6 m (i.e., 25 ft.) of tubing with an inner diameter of 6.4 mm (i.e., 0.25 in.) will hold 125 mL of water, which is an appropriate volume for use with a sterilization chamber having a volume of 42 L (i.e., 1.5 cu. ft.). It is preferred that the metal tubing is a heat-conductive metal tubing, e.g., copper tubing. As electric heating element 16 produces heat that is directly applied to the outer surface of inner cylindrical container 12 (to which it is fixed), some heat is radiated from electric heating element 16 into annular vacuum-insulated space 13 between the inner cylindrical container 12 and outer jacket 11, thereby raising the temperature of any air within annular chamber 13. The resulting beat is conducted through stainless steel outer jacket 11 and transferred to metal tubing 36, thereby pre-heating water contained therein prior to its transfer to sterilization chamber 14. It has been noted that, when the temperature of the walls of inner cylindrical container 12 has been raised to about 100° C. by electric heating element 16, the temperature of outer jacket 11 is raised to and maintained at about 40° C. to 60° C. by the heat radiating from electric heating element 16 into annular vacuum-insulated space 13. After the temperature within sterilization chamber 14 has reached 131° C., first and second water check valves 35a and 35b are opened concurrently with air solenoid valve 52 and air check valve 53, thereby providing a burst of compressed air to eject preheated water contained in the metal tubing 36 into sterilization chamber 14. After this has taken place, first and second water check valves 35a and 35b, air solenoid valve 52, air check valve 53 and vacuum solenoid valve 62 are closed. The water is heated by contact with the inner cylindrical container 12 thereby forming steam within sterilization chamber 14 while pressure is concurrently increased. When data communicated to PCI microcontroller 90 by the thermocouple 70 and pressure sensor 72 indicate that sterilization chamber 14 is completely saturated with steam, the temperature is stable at 131° C. and pressure has increased to say 24 inHg, a 4-min sterilization cycle is commenced during which the temperature and pressure values are maintained by direct communication and cooperation between PID temperature controller 91 and PIC microcontroller 90. During this time, the PID temperature controller is also communicating with thermocouple 70 and electric heating element 16, and PIC microcontroller is also communicating with humidity sensor 71, pressure sensor 72 and vacuum solenoid valve 62. The insulation provided to sterilization chamber 14 by vacuum-insulated annular space 13 in combination with the communication and cooperation between PID temperature controller 91 and PIC microcontroller 90 minimizes temperature fluctuations within the sterilizer chamber 14 during the sterilization period to about ±1° C. After the input time duration for sterilization has been completed, vacuum solenoid valve 62 is opened, thereby releasing pressurized steam from within sterilization chamber 14 through vacuum air line 64 interconnected through venturi device 57 within piping 56 of the second high-velocity airflow system. The steam then flows into condenser unit 58, where it is cooled into an air/water mixture, which is then separated into water and air within air/water separator apparatus 59. The water is expelled via water exit port 83 into water recycling line 61 for transfer to reservoir 30, and the air is expelled through air exit port 84 connected to air exhaust line 60.

When the pressure in sterilization chamber 14 has fallen to near ambient levels, drain solenoid valve 39 is opened, thereby allowing residual water condensed in sterilization chamber 14 to drain into drain line 40. Air solenoid valve 55 is then opened, providing a high-velocity flow of air through venturi device 57, thereby creating negative pressures, i.e., vacuum, applied to venturi tube portion 65 of vacuum line 64 and to venturi tube portion 41 of drain line 40 to remove all moisture from lines 40 and 64 and to apply negative pressures to sterilization chamber 14. Drain solenoid valve 39 is then closed and negative pressure is applied to sterilization chamber 14 through vacuum line 64 thereby removing all steam and moisture from sterilization chamber 14. Air solenoid valve 52, air check valve 53 and first and second water check valves 35a and 35b are then opened to provide a high-velocity flow of air through tubing 36 into sterilization chamber 14 under positive pressure for quickly drying and cooling sterilized items therein. The high-velocity airflow is removed from the sterilization chamber 14 through vacuum line 64 connected to venturi device 57 by venturi tip portion 65 for a preset period of drying time entered into user input electronic device 95. At the completion of the drying time, the table-top steam sterilizer of the present invention returns to the standby mode wherein drain solenoid valve 39 and vacuum solenoid valve 62 are held in open positions, while water solenoid valve 33, first and second water check valves 35a and 35b, air solenoid valves 52 and 55, and air check valve 53 are in held closed positions, after which, door 20 may be opened and sterilized items therein sterilization chamber 14 can be removed.

If so desired, the portable table-top steam sterilizer of the present invention can be provided with a removable one-piece molded or formed outer casing or alternatively, with a plurality of removable interconnecting outer panels for enclosing the double-walled vacuum-sealed vessel containing the sterilization chamber, the self-contained water supply infrastructure, the self-contained airflow piping infrastructure and the electronic controls. Those skilled in this art will understand that such casing or outer panels should be fitted with ports for receiving an electrical power supply, an external water supply and water waste lines with the water supply infrastructure, for connecting an external compressed air supply to the airflow piping infrastructure, and for connecting an air exhaust line. The casing or outer panel should provide an orifice for the sterilizer door to open therethrough or, alternatively, the casing or outer panel may be engaged with the flange protruding beyond the outer jacket of the double-walled vacuum-sealed vessel. The user input and output devices should be incorporated into the casing or outer panel.

It should be noted that the portable table-top steam sterilizer of the present invention can cooperate with a portable fuel-powered electric generator and portable supplies of compressed air such as compressed air tanks and portable fuel-powered air compressors or alternatively a portable air compressor powered by a portable electric generator. The water requirements may be met by the use of containerized water added periodically to the water reservoir. Consequently, the portable table-top steam sterilizer of the present invention is amenable for use in remote areas or in environments wherein hydro power and external water amenities are not available.

While this invention has been described with respect to the preferred embodiments, it is to be understood that various alterations and modifications can be made to components of the portable table-top steam sterilizer within the scope of this invention, which are limited only by the scope of the appended claims.

What is claimed is:

1. A portable table-top steam sterilization apparatus comprising:
a double-walled vacuum-insulated vessel provided with a sealingly engageable access door and defining therein a sterilization chamber,
an electric heating element interposed between walls of said double-walled;
a heat-conductive tubing contacting and coiled around an exterior of said vessel said tubing communicating with said sterilization chamber and also with a water supply;
an airflow piping infrastructure having an inlet and an outlet, said inlet connecting to an external supply of compressed air, said airflow piping infrastructure having 8 first airflow piping system adapted for delivery of a positive air pressure to said heat-conductive tubing, said airflow piping infrastructure also having a second airflow piping system adapted for delivery of a negative air pressure to said sterilization chamber, said second airflow system communicating with said outlet;
an air/water separator communicating with the airflow piping infrastructure outlet; and
a control system for controlling input of heat, water and airflow into said sterilization chamber to effect sterilization and subsequent cooling of contents positioned within said sterilization chamber.

2. The apparatus of claim 1 wherein the double-walled vacuum-insulated vessel comprises an inner cylindrical container disposed within an outer jacket, said inner cylindrical container being hermetically sealed under a negative pressure to said outer jacket, said inner container and outer jacket defining an annular vacuum-insulated space therebetween.

3. The apparatus of claim 2 wherein the electric heating element is in contact with an outer surface of said inner cylindrical container.

4. The apparatus of claim 3 wherein said electric heating element contacts only a bottom portion of said outer surface of said inner cylindrical container.

5. The apparatus of claim 1 wherein the water supply system comprises a water reservoir interconnected with a water outflow line communicating with said heat-conductive tubing.

6. The apparatus of claim 5 wherein said water outflow line is provided with a selonoid valve and a check valve.

7. The apparatus of claim 5 wherein the water reservoir is interconnected to said air/water separator.

8. The apparatus of claim 1 wherein the first airflow piping system is interconnected to said water outflow line.

9. The apparatus of claim 8 wherein said first airflow piping system is interconnected to said water outflow line via a check valve.

10. The apparatus of claim 1 wherein the second airflow system is provided with a venturi device for creating a negative air pressure therein, said venturi device being positioned for receiving therein mixtures of steam, water, and air from said sterilization chamber.

11. The apparatus of claim 10 wherein the second airflow system is provided with a vacuum line for communicating with said sterilization chamber.

12. The apparatus of claim 10 wherein the second airflow system is provided with a drain line for communicating with said sterilization chamber.

13. The apparatus of claim 1 wherein the air/water separator comprises an elongate casing equipped with a demountable cap, said casing and said cap sealingly engaging therein a cylindrical water filter having a bore therethrough along a longitudinal axis, said cap having a port therethrough for communicating with said bore in said water filter, said elongate casing having a first bore therethrough for releasing air therefrom and a second bore therethrough for expelling water therefrom.

14. An air/water separator for cooperating with exhaust outlets of a steam sterilizer, the separator comprising an elongate casing sealingly engaging therein a cylindrical water filter having a bore therethrough along a longitudinal axis, said casing having a first port therethrough for communicating with said bore in said water filter, a second bore therethrough for releasing air therefrom and a third bore therethrough for expelling water therefrom.

15. The separator of claim 14 wherein said elongate casing is provided with a demountable cap, said cap having a port therethrough for communicating with said bore in said water filter.

16. A method for sterilizing items, the method comprising;
securing an item to be sterilized into a sterilization chamber contained within a double-walled vacuum-insulated vessel;
concurrently applying heat to the inner wall of said vessel and to water contained within a conduit contiguously engaged with the outer wall of said vessel;
transferring said heated water with a positive air pressure to said heated vessel, converting said water to steam within the sterilization chamber therein, and maintaining said steam for a selected period of time;
supplying a negative air pressure to said sterilization chamber thereby exhausting said steam from said sterilization chamber;
additionally supplying a positive air pressure to said sterilization chamber thereby providing a turbulent cooling drying air flow therethrough the sterilization chamber;
releasing said positive and negative air pressures thereby returning the sterilization chamber to an ambient condition; and
removing said items from within said double-walled vacuum-insulated vessel.

17. A steam sterilization method comprising the steps of:
Placing objects to be sterilized in a double walled container having a cavity between the two walls evacuated and permanently sealed, and controlling the temperature inside the container to achieve complete sterilization.
